(19) United States
(12) Patent Application Publication
(10) Pub. No.: US 2024/0192204 A1
(43) Pub. Date: Jun. 13, 2024

(54) A DEVICE AND ASSAYS FOR DETECTION OF PATHOGENS

(71) Applicant: Universal Sequencing Technology Corporation, Canton, MA (US)

(72) Inventors: Peiming Zhang, Gilbert, AZ (US); Ming Lei, Sharon, MA (US); Sanjay B. Hari, Sharon, MA (US)

(73) Assignee: Universal Sequencing Technology Corporation, Canton, MA (US)

(21) Appl. No.: 17/998,556

(22) PCT Filed: May 17, 2021
(86) PCT No.: PCT/US2021/032809

§ 371 Date: Nov. 11, 2022

Related U.S. Application Data
(60) Provisional application No. 63/025,914, filed on May 15, 2020.

(51) Int. Cl. G01N 33/543; G01N 33/553; G01N 33/569

(52) U.S. Cl. CPC G01N 33/54388; G01N 33/54346; G01N 33/553; G01N 33/56983; G01N 2333/165

(57) ABSTRACT

This invention provides a device and assays for the detection of pathogens.

This application claims priority to U.S. Provisional Application Ser. No. 63/025914 filed May 15, 2020, the entire disclosures of which are hereby incorporated herein by reference.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present disclosure, suitable methods and materials are described below. The materials, processes, and examples are illustrative only and are not intended to be limiting. All publications, patents, and other documents mentioned herein are incorporated by reference in their entirety.

### FIELD

Embodiments of the present invention are related to biomolecular detection using a lateral flow device.

### BACKGROUND OF THE INVENTION

Since its first outbreak in Wuhan, China, in December of 2019, SARS-CoV-2 has infected more than four million people worldwide, resulting in more than 300000 deaths from the COVID-19 disease as of May 15, 2020. The PCR-based nucleic acid test has been a primary choice for the detection of SARS-CoV-2 due to its sensitivity¹. However, it requires highly trained technical staff, sophisticated equipment (costing USD 20K-50K), and certain facilities. The PCR test protocol is also complex, which is mainly suitable for large, centralized diagnostic laboratories. It typically takes 4-6 hours to complete a test, but the logistical requirement to ship clinical samples means the turnaround time is 24 hours at best.² These factors contribute to longer turnaround time, staffing costs, capital, consumables, and risks of both carryover contamination and biosafety when handling clinical samples. Besides, the PCR alone is not able to determine the infectivity of the disease effectively.

Amid the COVID-19 pandemic, numerous lateral flow assays (LFA) have been applied to the rapid detection of antibodies^{3, 4}, viral RNA^{5, 6}, and antigen⁷ of SARS-CoV-2. LFA is a chromatographic technique for disease detection with many advantages: low cost, ease of use, rapid on-site response, and visual readout.⁸ LFAs are particularly useful for expecting to achieve quick results, making them ideal for point-of-care (POC) applications. Unfortunately, present LFA-based tests have delivered unsatisfactory results compared to the gold standard ELISA, which may be attributed to their unsophisticated surface chemistry of those products.

An LFA is generally performed on a test strip assembled in a cartridge. As shown in FIG. 1, the test strip consists of a sample pad for loading sample, a conjugate pad for storing labeled affinity probes, a reaction substrate (typically a thin nitrocellulose membrane) where molecular interactions take place and an absorbent pad for waste absorption. Adding a buffer solution onto the sample pad would drive analytes to flow through the substrate towards the absorbent pad by capillary forces. The target molecules are captured by an affinity probe immobilized in a test line to form a sandwich structure with a second affinity probe attached to a signaling moiety, such as gold nanoparticles, for colorimetric readout. Note that a control line is also built in the test strip to ensure a quality test. The success of LFA depends to a great extent on how the affinity reagents are immobilized on the reaction substrate and gold nanoparticles. Usually, for the conventional LFA, an affinity probe, such as an antibody, is spotted onto the nitrocellulose surface. It adheres by physical adsorption through noncovalent interactions, such as Van der Waals forces, hydrogen bonding, and hydrophobic interactions. The primary issue with the physical adsorption is that those adsorbed molecules could disassociate from the surface⁹ or be readily displaced by molecules from the sample solution (Vroman effect).¹⁰ These effects would reduce the detection sensitivity and cause a product reliability issue. For detecting viral RNA, a DNA capture probe is usually biotinylated and attached to streptavidin physically adsorbed on the nitrocellulose (NC) membrane surface.¹¹ This method still relies on physical adsorption of the streptavidin molecule, subjected to the same limitation described above. Alternatively, a DNA probe can be immobilized on the surface through UV crosslinking.¹² However, this process still results in random probe orientation, preventing the probe from hybridizing to targets effectively and leading to reduced detection sensitivity. Due to the continuation of the coronavirus pandemic worldwide, a reliable rapid LFA test product with high sensitivity and specificity is urgently needed.

### BRIEF DESCRIPTION OF THE DRAWINGS

FIG. 1: Schematic illustration of a general configuration for the lateral flow test.

FIG. 2: Schematic illustration of a lateral flow test strip bearing hydrogel polymer embedded lines for immobilization of affinity molecules.

FIG. 3: (A) Process of fabricating hydrogel polymer lines on a nitrocellulose membrane by photolithography; (B) Method to functionalize the hydrogel polymer lines with affinity molecules

FIG. 4: Detecting virus RNA by a ligation-rolling circle amplification (LRCA) assay (LRCA) based on a padlock DNA probe. (A) Sample preparation; (B) LFA of RNA on the hydrogel patterned test strip.

FIG. 5: (A) Process of preparing DNA nano-balls with gold nanoparticles; (B) LFA of antigens on the hydrogel patterned test strip.

FIG. 6: A procedure of modifying antibodies for their immobilization and attachment. (i) Oxidation of Antibody (Ab) with sodium periodate; (ii) BCN modification of Ab.

FIG. 7: Structures of molecules for the formation of a mixed monolayer.

FIG. 8: LFA of antibodies on the hydrogel patterned test strip

FIG. 9: Sequence of Streptavidin-sso7d fusion protein

### SUMMARY OF THE INVENTION

This invention provides a lateral flow assay (LFA) test strip platform for virus detection and infectious disease diagnosis based on the viral nucleic acids, antigens, and antibodies. Although the test strip is primarily intended to use for virus detection, especially SARS-CoV-2 virus or COVID-19 diagnosis, it can be extended to detect any other viruses and pathogens, and the related diseases. By keeping these in mind, the following embodiments present the invention by detecting SARS-CoV-2 as an example.

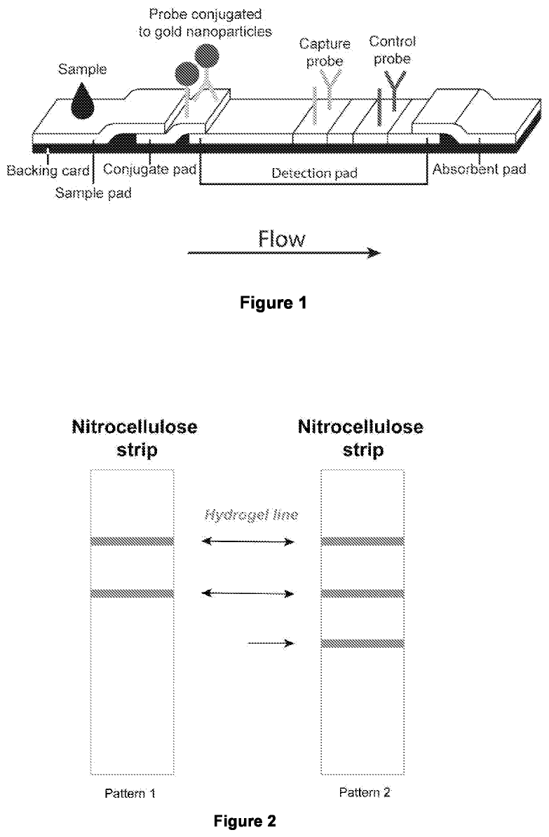

This invention provides a method to fabricate test lines with hydrogel polymer in an LFA test strip. As shown in FIG. 2, each test line bears the hydrogel polymer in the lateral flow test strip, which provides a biologically friendly environment for interactions of an affinity molecule with its cognate. The hydrogel polymer allows for the immobilization of affinity molecules by chemical attachment or physical containment, reducing the risk of affinity molecules desorbing from the surface, which is a major drawback of traditional physical adsorption. The hydrogel polymer also allows the affinity molecules to be immobilized with a predefined orientation on the surface, rendering them to interact with target molecules more effectively, meaning higher sensitivity.

In some embodiments, the invention provides a process of fabricating the hydrogel polymer embedded test lines on a test strip by photolithography.

In some embodiments, the invention provides examples for utilizing the hydrogel polymer patterned lateral flow test strips to detect coronaviruses. Streptavidin is embedded in hydrogel to improve the immobilization of affinity probes attached to the test line or the control line. In some embodiments, the invention provides a fusion protein consisting of streptavidin from Streptomyces avidinii genetically fused to a small non-specific DNA-binding protein, for example, sso7d from Saccharolobus solfataricus. The fusion protein can be used in place of the streptavidin embedded in the hydrogel for even better immobilization effects.

### DETAILED DESCRIPTION

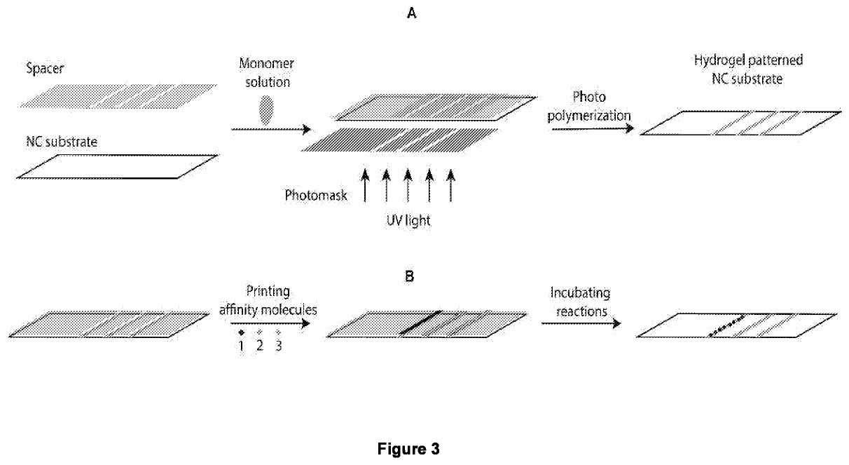

In one embodiment, the invention provides a photolithographic process for fabricating polyacrylamide hydrogel lines in a nitrocellulose (NC) membrane. As illustrated in FIG. 3A, a patterned spacer is first placed on the NC membrane, followed by coating it with a thin film of a mixed solution comprising acrylamide and bisacrylamide plus a photoinitiator. Here, the ratio of bisacrylamide is about 0% to 10%, preferably ≤5%, and most preferably about ≤1%; the photoinitiator is about 0.01% to 10% of total acrylamide, preferably 0.1% to 1%. The film is irradiated from the bottom by UV light with a photomask to form polyacrylamide hydrogel lines (or pads) at predefined locations. Following the completion, the polyacrylamide can be activated by hydrazine for attaching biomolecules containing aldehyde and ketone functions.¹³ In the same way, the polyacrylamide hydrogel lines can be fabricated to include an NHS ester by the addition of N-acryloxysuccinimide into the acrylamide solution mentioned above. Thus, amino-functionalized biomolecules can be attached to the hydrogel lines.

In another embodiment, the mixed solution of hydrogel monomers and photoinitiator such as those mentioned in the above embodiment is printed on the NC substrate in a well-controlled manner so that no photomask is needed for the UV light irradiation to form the hydrogel polymer lines or pads.

In some embodiments, the hydrogel polymer lines are printed or spotted directly onto the NC membrane, and the hydrogel polymerization is initiated by chemicals other than photoinitiators, such as ammonium persulfate (APS) and tetramethylethylenediamine (TEMED) for polyacrylamide hydrogels, or is initiated by heating instead of chemical initiators, such as agarose hydrogels.

In some embodiments, the LFA membrane comprises cellulose acetate, polyvinylidene fluoride (PVDF), charge-modified nylon, polyethersulfone (PES), fibers, or other porous materials that can cause wicking or capillary flow effects.

In one embodiment, the invention provides a method to print affinity molecules to the hydrogel lines using a nozzle dispenser, as illustrated in FIG. 3B. The biomolecule is attached to the surface through a chemical reaction either covalently or noncovalently.

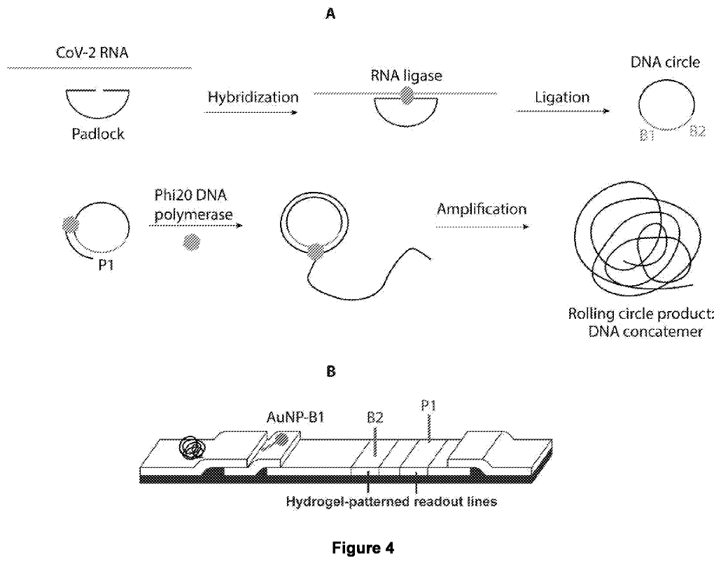

In some embodiments, the invention provides methods to detect viral nucleic acids. FIG. 4 shows an assay for detecting viral RNA using a ligation-rolling circle amplification (LRCA) assay. Unlike PCR that requires repeated thermal cycling, RCA is an isothermal, room temperature-based amplification technique. Recent progress has made RCA a limit-of-detection of sub-ten aM.¹⁴ For detecting the viral RNA, a padlock probe is first hybridized to its RNA target and then ligated into a circle by either DNA or RNA ligase (FIG. 4A). After digesting the linear DNA by nuclease, the circle DNA is used as a template to generate a DNA concatemer with a length of kilo-bases, for example, by a Phi29 DNA polymerase. For LFA, the padlock is designed to bear two barcodes B1, and B2 (FIG. 4B), which are designed to recognize the products from LRCA. P1 can be used as a primer of DNA polymerase and a signal probe to be conjugated to gold nanoparticles in the control line. The padlock based method also allows multiplex detection of RNA¹⁵

In some embodiments, the invention introduces a universal base to the padlock probe at proximity to the nick site for improving the efficiency of the ligation. A universal base can form base pairs with naturally occurring nucleobases indiscriminately. For example, the universal base 3-nitropyrrole increases the fidelity and rate of ligation chain reaction (PCR).¹⁶ It has been used in PCR and Sanger sequencing primers, and up to nine universal bases were used in a 17 base primer for the Sanger sequencing.¹⁷

In some embodiments, the universal base is put in the padlock probe with its location close to the site where the mutation occurs with a high frequency in the target to maintain the assay to have high sensitivity to variants of the virus from different samples.

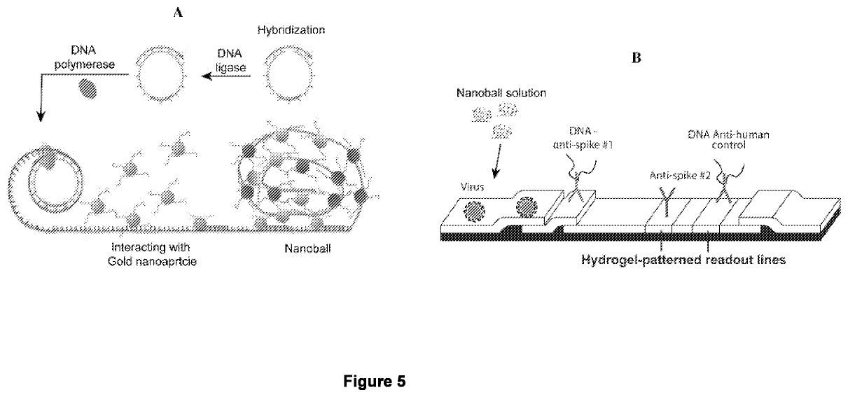

In one embodiment, the invention provides a DNA-gold nano-ball to detect SARS-CoV-2 antigens on the hydrogel polymer test strip. As shown in FIG. 5A, the nano-ball is composed of a DNA concatemer complexing with gold nanoparticles labeled with multiple DNA probes each, which can be prepared by LRCA using a procedure reported in the literature.¹⁸

In some embodiments, the invention provides an assay to detect viral antigens on the hydrogel patterned test strip. As shown in FIG. 5B, the LFA starts with immobilizing the primary antibody (anti-spike #2) to the test line, and the secondary antibody (anti-spike #1) is labeled with DNA probes that are complementary to DNA attached to gold nanoparticles of the nanoball, and preloaded in the conjugate pad. The virus sample from patient swabs is added to the sample pad and then developed with the nanoball solution. The virus is first moved to the conjugate pad to form a complex with anti-spike #1 and then captured by anti-spike #2 on the test line. Meanwhile, the nanoball moves and interact with anti-spike #1 on the test line to signal the existence of the virus.

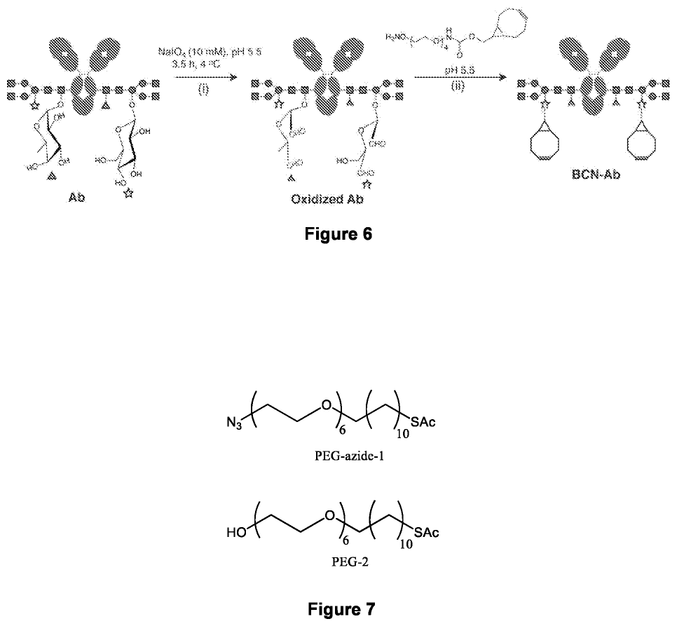

In one embodiment, the invention provides a method to modify antibodies for their immobilization and attachment. FIG. 6 shows the process of modifying antibodies. First, the antibody is oxidized with sodium periodate to create aldehyde groups at its Fc domain. That allows the oxidized antibody to react readily with the hydrazine-activated polyacrylamide. The oxidized antibody is further functionalized with cyclooctyne for conjugating to DNA (FIG. 6). The antibody BCN-Ab reacts with azide functionalized DNA spontaneously.

In some embodiments, the invention provides a method to form a mixed monolayer on gold nanoparticles to prevent non-specific adsorption. FIG. 7 shows the structures of these chemical reagents. PEG-azide-1 functions as a linker for the molecular attachment, and PEG-2 forms a monolayer on the gold surface for preventing the non-specific adsorption.¹⁹

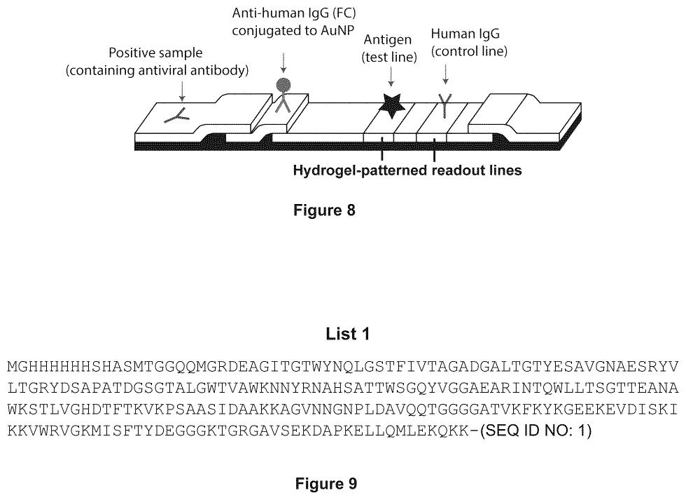

In some embodiments, the invention provides an assay to detect an anti-virus antibody on the hydrogel polymer patterned test strip. As shown in FIG. 8, the LFA starts with immobilizing an antigen to the test line and loading anti-human IgG (Fc) antibody labeled with a gold nanoparticle in the conjugate pad. The serum sample from a patient containing an anti-virus antibody is added to the sample pad and then developed with a chase buffer. The anti-virus antibody is first moved to the conjugate pad to form a complex with anti-human IgG antibody and then captured by antigen on the test line. The test line is lit up for a positive result.

In some embodiments, streptavidin is contained in the said lines by photopolymerizing an acrylamide solution containing streptavidin under the condition mentioned above. Next, biotinylated affinity molecules are deposited at the exact locations for LFA to detect the targeting molecules.

In other embodiment, the above said streptavidin is expressed as a genetic fusion with a small DNA-binding protein, for example, sso7d from Saccharolobus solfataricus (List 1) for the photopolymerization.

### General Remarks

While the present invention has been illustrated by a description of various embodiments and while these embodiments have been described in considerable details, it is not the intention of the applicants to restrict or in any way limit the scope of the appended claims to such detail.

Additional advantages and modifications will readily appear to those skilled in the art. The invention in its broader aspects is therefore not limited to the specific details, representative apparatus and method, and illustrative example shown and described. Accordingly, departures may be made from these details without departing from the spirit or scope of applicant's general inventive concept.

### Reference

1. Gudbjartsson, D. F.; Helgason, A.; Jonsson, H.; Magnusson, O. T.; Melsted, P.; Norddahl, G. L.; Saemundsdottir, J.; Sigurdsson, A.; Sulem, P.; Agustsdottir, A. B.; Eiriksdottir, B.; Fridriksdottir, R.; Gardarsdottir, E. E.; Georgsson, G.; Gretarsdottir, O. S.; Gudmundsson, K. R.; Gunnarsdottir, T. R.; Gylfason, A.; Holm, H.; Jensson, B. O.; Jonasdottir, A.; Jonsson, F.; Josefsdottir, K. S.; Kristjansson, T.; Magnusdottir, D. N.; le Roux, L.; Sigmundsdottir, G.; Sveinbjornsson, G.; Sveinsdottir, K. E.; Sveinsdottir, M.; Thorarensen, E. A.; Thorbjornsson, B.; Löve, A.; Masson, G.; Jonsdottir, I.; Möller, A. D.; Gudnason, T.; Kristinsson, K. G.; Thorsteinsdottir, U.; Stefansson, K., Spread of SARS-CoV-2 in the Icelandic Population. New England Journal of Medicine 2020, 382, 2302-2315
2. Sheridan, C., Fast, portable tests come online to curb coronavirus pandemic. Nature Biotechnology 2020, 38, 509-522.
3. Adams, E. R.; Anand, R.; Andersson, M. I.; Auckland, K.; Baillie, J. K.; Barnes, E.; Bell, J.; Berry, T.; Bibi, S.; Carroll, M.; Chinnakannan, S.; Clutterbuck, E.; Cornall, R. J.; Crook, D. W.; De Silva, T.; Dejnirattisai, W.; Dingle, K. E.; Dold, C.; Eyre, D. W.; Farmer, H.; Hoosdally, S. J.; Hunter, A.; Jeffrey, K.; Klenerman, P.; Knight, J.; Knowles, C.; Kwok, A. J.; Leuschner, U.; Liu, C.; Lopez-Camacho, C.; Matthews, P. C.; McGivern, H.; Mentzer, A. J.; Milton, J.; Mongkolsapaya, J.; Moore, S. C.; Oliveira, M. S.; Pereira, F.; Peto, T.; Ploeg, R. J.; Pollard, A.; Prince, T.; Roberts, D. J.; Rudkin, J. K.; Screaton, G. R.; Semple, M. G.; Skelly, D. T.; Smith, E. N.; Staves, J.; Stuart, D.; Supasa, P.; Surik, T.; Tsang, P.; Turtle, L.; Walker, A. S.; Wang, B.; Washington, C.; Watkins, N.; Whitehouse, J.; Beer, S.; Levin, R.; Espinosa, A.; Georgiou, D.; Martinez Garrido, J. C.; Thraves, H.; Perez Lopez, E.; del Rocio Fernandez Mendoza, M.; Sobrino Diaz, A. J.; Sanchez, V., Evaluation of antibody testing for SARS-CoV-2 using ELISA and lateral flow immunoassays. medRxiv 2020, 2020.04.15.20066407.
4. Whitman, J. D.; Hiatt, J.; Mowery, C. T.; Shy, B. R.; Yu, R.; Yamamoto, T. N.; Rathore, U.; Goldgof, G. M.; Whitty, C.; Woo, J. M.; Gallman, A. E.; Miller, T. E.; Levine, A. G.; Nguyen, D. N.; Bapat, S. P.; Balcerek, J.; Bylsma, S. A.; Lyons, A. M.; Li, S.; Wong, A. W.-y.; Gillis-Buck, E. M.; Steinhart, Z. B.; Lee, Y.; Apathy, R.; Lipke, M. J.; Smith, J. A.; Zheng, T.; Boothby, I. C.; Isaza, E.; Chan, J.; II, D. D. A.; Lee, J.; Macrae, T. A.; Kyaw, T. S.; Wu, D.; Ng, D. L.; Gu, W.; York, V. A.; Eskandarian, H. A.; Callaway, P. C.; Warrier, L.; Moreno, M. E.; Levan, J.; Torres, L.; Farrington, L. A.; Loudermilk, R.; Koshal, K.; Zorn, K. C.; Garcia-Beltran, W. F.; Yang, D.; Astudillo, M. G.; Bernstein, B. E.; Gelfand, J. A.; Ryan, E. T.; Charles, R. C.; lafrate, A. J.; Lennerz, J. K.; Miller, S.; Chiu, C. Y.; Stramer, S. L.; Wilson, M. R.; Manglik, A.; Ye, C. J.; Krogan, N. J.; Anderson, M. S.; Cyster, J. G.; Ernst, J. D.; Wu, A. H. B.; Lynch, K. L.; Bern, C.; Hsu, P. D.; Marson, A., Test performance evaluation of SARS-CoV-2 serological assays. medRxiv 2020, 10.1101/2020.04.25.20074856.
5. Broughton, J. P.; Deng, X.; Yu, G.; Fasching, C. L.; Servellita, V.; Singh, J.; Miao, X.; Streithorst, J. A.; Granados, A.; Sotomayor-Gonzalez, A.; Zorn, K.; Gopez, A.; Hsu, E.; Gu, W.; Miller, S.; Pan, C.-Y.; Guevara, H.; Wadford, D. A.; Chen, J. S.; Chiu, C. Y., CRISPR-Cas12-based detection of SARS-CoV-2. Nature Biotechnology 2020. 38. 870-874.
6. Rauch, J. N.; Valois, E.; Solley, S. C.; Braig, F.; Lach, R. S.; Baxter, N. J.; Kosik, K. S.; Arias, C.; Acosta-Alvear, D.; Wilson, M. Z., A Scalable, Easy-to-Deploy, Protocol for Cas13-Based Detection of SARS-CoV-2 Genetic Material. bioRxiv 2020, 2020.04.20.052159.
7. Vandenberg, O., Development and potential usefulness of the COVID-19 Ag Respi-Strip diagnostic assay in a pandemic context. medRxiv 2020, 2020.04.24.20077776.
8. Bahadιr, E. B.; Sezgintürk, M. K., Lateral flow assays: Principles, designs and labels. TrAC Trends in Analytical Chemistry 2016, 82, 286-306.
9. Holstein, C. A.; Keniston, K.; Fu, E.; Chevalier, A.; Olsen, C.; Baker, D.; Yager, P.; Bennett, S.; Li, B., Immobilizing affinity proteins to nitrocellulose: a toolbox for paper-based assay developers. Anal Bioanal Chem 2016, 408, 1336-46.
10. Hirsh, S. L.; Mckenzie, D. R.; Nosworthy, N. J.; Denman, J. A.; Sezerman, O. U.; Bilek, M. M., The Vroman effect: competitive protein exchange with dynamic multilayer protein aggregates. Colloids Surf B Biointerfaces 2013, 103, 395-404.
11. Gao, X.; Xu, H.; Baloda, M.; Gurung, A. S.; Xu, L. P.; Wang, T.; Zhang, X.; Liu, G., Visual detection of microRNA with lateral flow nucleic acid biosensor. Biosens Bioelectron 2014, 54, 578-84.
12. Rohrman, B. A.; Leautaud, V.; Molyneux, E.; Richards-Kortum, R. R., A Lateral Flow Assay for Quantitative Detection of Amplified HIV-1 RNA. PLOS ONE 2012, 7, e45611.
13. Arenkov, P.; Kukhtin, A.; Gemmell, A.; Voloshchuk, S.; Chupeeva, V.; Mirzabekov, A., Protein Microchips: Use for Immunoassay and Enzymatic Reactions. Analytical Biochemistry 2000, 278, 123-131.
14. Park, K. W.; Lee, C. Y.; Batule, B. S.; Park, K. S.; Park, H. G., Ultrasensitive DNA detection based on target-triggered rolling circle amplification and fluorescent poly(thymine)-templated copper nanoparticles. RSC Advances 2018, 8, 1958-1962.
15. Ciftci, S.; Neumann, F.; Hernández-Neuta, I.; Hakhverdyan, M.; Bálint, Á.; Herthnek, D.; Madaboosi, N.; Nilsson, M., A novel mutation tolerant padlock probe design for multiplexed detection of hypervariable RNA viruses. Scientific reports 2019, 9, 2872.
16. Luo, J.; Bergstrom, D. E.; Barany, F., Improving the Fidelity of Thermus Thermophilus DNA Ligase. Nucleic Acids Research 1996, 24, 3071-3078.
17. Nichols, R.; Andrews, P. C.; Zhang, P.; Bergstrom, D. E., A universal nucleoside for use at ambiguous sites in DNA primers. Nature 1994, 369, 492-493.
18. Hu, B.; Guo, J.; Xu, Y.; Wei, H.; Zhao, G.; Guan, Y., A sensitive colorimetric assay system for nucleic acid detection based on isothermal signal amplification technology. Anal. Bioanal. Chem. 2017, 409, 4819-4825.
19. Senapati, S.; Biswas, S.; Manna, S.; Ros, R.; Lindsay, S.; Zhang, P., A Y-Shaped Three-Arm Structure for Probing Bivalent Interactions between Protein Receptor-Ligand Using AFM and SPR. Langmuir 2018, 34, 6930-6940.

1. A system for the detection or identification of an analyte comprising a lateral flow assay (LFA) test strip, wherein the test strip comprises one or more test lines and one or more control lines, wherein at least one of the test lines or control lines comprises a hydrogel pattern.

2. The system of claim 1, wherein the test strip comprises a substrate, a sample pad, a probe conjugate pad, a detection pad, and an absorbent pad, and wherein the detection pad comprises the test line and the control line, and each of the pads comprises a wicking material.

3. The system of claim 2, wherein the wicking material is selected from the group consisting of cellulose acetate, nitrocellulose, polyvinylidene fluoride (PVDF), charge-modified nylon, polyethersulfone (PES), glass fiber, a porous material, a fibrous material, and a combination thereof.

4. The system of claim 2 further comprising
a. a first affinity probe on the conjugate pad configured to capture the analyte;
b. a second affinity probe on the test line configured to capture the conjugated analyte; and
c. a third affinity probe on the control line configured to capture the first affinity probe.

5. The system of claim 1 further comprises a cartridge, wherein the test strip is assembled or enclosed in the cartridge.

6. The system of claim 1, wherein the analyte comprises a pathogen or a component of the pathogen of either viral or bacterial origin or a combination thereof.

7. The system of claim 1, wherein the analyte is a biomolecule selected from the group consisting of a nucleic acid, a fragment of a viral or a bacterial RNA or DNA, a protein, an antibody, an antigen, a carbohydrate, and a combination thereof.

8. The system of claim 1, wherein the hydrogel patterned line is fabricated by photolithography or is printed or spotted on the test strip.

9. The system of claim 4, wherein the affinity probe is a biomolecule selected from the group consisting of a nucleic acid, an oligo, a protein, a peptide, an antibody, an antigen, a carbohydrate, either natural, synthesized or modified, and a combination thereof.

10. The system of claim 4, wherein the first affinity probe is attached to a gold nanoparticle, either covalently or non-covalently.

11. The system of claim 10, wherein the gold nanoparticle is covered by a monolayer of PEG molecules.

12. The system of claim 10, wherein the first affinity probe is attached to the gold nanoparticle through a linker, wherein the linker comprises a PEG molecule.

13. The system of claim 9, wherein the antibody is modified with a sodium periodate and a BCN.

14. The system of claim 1, wherein the hydrogel patterned line is made of poly(acrylamide) or poly(bisacrylamide) or a combination thereof, wherein the hydrogel patterned line comprises an NHS ester.

15. (canceled)

16. The system of claim 1, wherein the hydrogel patterned line comprises a streptavidin or a fusion protein or a combination thereof, either natural, synthesized or modified, wherein the fusion protein comprises a streptavidin fused to a small non-specific DNA-binding protein.

17. (canceled)

18. The system of claim 1, wherein each test line or each control line is functionalized with an affinity probe, wherein the affinity probe is a biomolecule selected from the group consisting of a nucleic acid, an oligo, a protein, a peptide, an antibody, an antigen, a carbohydrate, either natural, synthesized or modified, and a combination thereof.

19. The system of claim 1, wherein the analyte is identified by a colorimetric readout.

20. The system of claim 1, wherein the analyte is a viral RNA and is amplified by a ligation-rolling circle amplification (LRCA) based on a padlock probe.

21. The system of claim 20, wherein the padlock probe is modified by including a universal base.

22. The system of claim 21, wherein the universal base comprises a 3-nitropyrrole.

23-48.(canceled)


SEQUENCE LISTING (1 sequence)

SEQ ID NO: 1 - protein, 236 aa, Artificial Sequence
  Description of Artificial Sequence Streptavidin-sso7d fusion protein
MGHHHHHHSHASMTGGQQMGRDEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQTGGGGATVKFKYKGEEKEVDISKIKKVWRVGKMISFTYDEGGGKTGRGAVSEKDAPKELLQMLEKQKK